(19) United States
(12) Patent Application Publication
(10) Pub. No.: US 2008/0300262 A1
(43) Pub. Date: Dec. 4, 2008

(54) Combination Therapy for Relief of Pain

(76) Inventor: Terrance P. Snutch, Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 11/910,909

(22) PCT Filed: Apr. 10, 2006
(86) PCT No.: PCT/CA06/00545

§ 371 Date: Jul. 29, 2008

Related U.S. Application Data
(60) Provisional application No. 60/669,820, filed on Apr. 8, 2005.

(51) Int. Cl. A61K 31/495; A61K 31/445; A61P 25/00

(52) U.S. Cl. 514/255.04; 514/331

(57) ABSTRACT

The present invention relates to analgesic compositions comprising a non-peptidyl N-type calcium channel blocker, and at least one other analgesic compound, and to methods of using N-type calcium channel blockers in combination with at least one other pain-relieving compound that alleviates pain through a mechanism other than N-type calcium channel blockage, or with a non-pharmacological therapeutic protocol.

### TECHNICAL FIELD

This invention relates to compositions and methods useful for treating pain. It includes compositions having an active component that is an N-type calcium channel blocker combined with an active component that operates by a different mechanism. A preferred class of N-type calcium channel blockers is identified. The invention also relates to methods for treating pain, which include concurrently treating a subject in need of pain relief with an N-type calcium channel blocker and at least one pain-relieving therapeutic compound or non-pharmacological protocol that operates by a mechanism other than blocking N-type calcium channels.

### BACKGROUND ART

N-type calcium channels are mainly localized in neurons, and compounds called channel blockers that inhibit ion transport through these channels have been described as treatments for a variety of conditions including pain. Certain peptidyl compounds that block N-type calcium channels are known to alleviate pain. For example, ziconotide (Prialt™) is a synthetic version of a peptidyl natural product that has been shown to inhibit N-type calcium channels and is used to treat severe, chronic pain. See “Ziconotide: Neuronal Calcium Channel Blocker for Severe Chronic Pain,” G. P. Miljanich' Current Medicinal Chemistry, vol. 11(23), pp. 3029-3040 (December 2004). See also Textbook of Pain, 4^{th} ed., P. D. Wall and R. Melzack, ed., Elsevier Science, pg. 1236 (2003); and the FDA label information for Prialt™, available online at www.fda.gov/cder/foi/label/2004/021060lbl.pdf. Ziconotide can reduce the amount of opioid pain medication required by its users; however, it suffers from severe bioavailability problems and cannot be administered orally or even by typical intramuscular or intravenous injection. As a result, it is administered only intrathecally, by a pumping device that slowly delivers it directly to the dorsal horn in the spinal cord, where it is believed to act by binding to the N-type calcium channels located on the primary nociceptive (A-δ and C) afferent nerves in the superficial layers (Rexed laminae I and II) of the dorsal horn. Even though ziconotide is delivered directly to the spinal cord, it is often used in combination with other extremely potent pain relievers such as opioids. Furthermore, its use is appropriate only for a very narrow category of severe chronic pain problems that are refractory to other known medications.

Other peptide-derived N-type calcium channel blockers have also been reported to be capable of treating pain. Thus U.S. Pat. No. 6,605,608 discloses amino acid derivatives that are active N-type calcium channel blockers. Likewise, U.S. Pat. Nos. 6,362,174 and 6,323,243 disclose compounds described as reduced dipeptide and tyrosine-derived N-type calcium channel blockers, respectively; thus these compounds, too, may be considered peptide derivatives, though they include some analogs that have been modified so that they no longer closely resemble a peptide, e.g. they are not N-acylated alpha-amino acid derivatives. U.S. Pat. No. 6,251,918 discloses similar compounds that are described as aniline derivatives, and that contain either an alpha-amino acid or a reduced version of one.

Certain other N-type calcium channel blockers are also known, including quinoline compounds disclosed in U.S. Pat. Nos. 6,815,447 and 6,841,680. U.S. Pat. No. 6,610,717 discloses non-peptidyl compounds that are dihydropyridine derivatives described as selective N-type calcium channel blockers for treating pain. U.S. Pat. No. 6,218,538 discloses a family of dihydropyrimidines that are also described as selective N-type calcium channel blockers which are useful to treat pain, among other things.

U.S. Pat. No. 6,267,945 teaches that certain farnesol-related compounds have selective activity as N-type calcium channel blockers, and are thus useful for treating stroke and pain as well as other calcium-related conditions.

U.S. Pat. Nos. 6,011,035; 6,294,533; 6,310,059; 6,387,897, and 6,617,322 and published U.S. Patent Applications No. 2003/0045530, US2003/0199523, US2004/0034035, US2004/0044004, US2004/0147529, US2004/0180323, US2004/0192703, US2004/0209872, US2004/0259866, US2004/0266784, and US2005/0014748, each of which is specifically incorporated by reference, describe selective N-type calcium channel blockers that were designed based on the recognition that the combination of a nitrogen-containing central ring coupled through a linker to a benzhydryl or isosteric moiety results in effective calcium channel blocking activity. The compounds disclosed in these patents and applications generally include a benzhydryl group or a saturated or partially saturated benzhydryl group that is linked to a 5-6 membered nitrogen-containing saturated central ring. Another ring-containing group is also linked to the central ring, which is typically a pyrrolidine, piperazine or piperidine; often it is a 1,4-disubstituted piperidine or 1,4-disubstituted piperazine.

The compounds within the listed patents and applications that have a 5-6 membered nitrogen-containing saturated central ring that is linked to a benzhydryl or a partially or fully saturated benzhydryl group and to another group containing at least one additional ring are referred to herein as tetracyclic or pentacyclic channel blockers. These compounds are particularly useful for treating acute and chronic pain. However, their combinations with pain treatments having a mechanism of action different from N-type calcium channel blocking and the advantages provided by such combinations have not been described.

### DISCLOSURE OF THE INVENTION

The present invention is based on the observation that the pain-relieving effect of an N-type calcium channel blocking compound is enhanced when combined with the pain-relieving effects of compounds and treatments that operate by mechanisms other than blocking N-type calcium channels. Thus N-type calcium channel blockers can be combined with other pain-relieving therapies to provide enhanced pain relief, and the combination minimizes the risks associated with increased reliance on a single type of pain relieving medication. For example, excessive use of opioids can lead to dependence and tolerance; the known selective COX-2 inhibitors are suspected to increase the risk of adverse cardiac events; most compounds that inhibit both COX-1 and COX-2 have anticoagulant activity and tend to cause gastrointestinal bleeding, and other analgesics such as acetaminophen often exhibit side effects such as hepatotoxicity. These factors thus frequently limit the dosages that are safely feasible for any one analgesic.

The present invention provides novel compositions and methods for alleviating pain which employ such N-type calcium ion channel blockers in combination with another pain relief treatment such as an NSAID, or a compound which selectively inhibits COX-2, or an opioid, or an adjuvant analgesic such as an antidepressant. Such compositions are useful for the preparation of a medicament, especially one for the treatment of pain in a subject, typically a human subject, in need of such treatment.

The present invention provides methods for treating pain that include administering a first compound that alleviates pain by blocking N-type calcium channels and administering a second therapy that alleviates pain by a mechanism other than blocking N-type calcium channels. The second therapy may be a second compound admixed with the first compound, or it may be a second compound formulated as a separate composition, or it may be a non-pharmacological treatment such as transcutaneous electrical nerve stimulation (TENS) or percutaneous electrical nerve stimulation (PENS). The first compound and the second therapy may be administered together or they may be administered separately, but separate administration is within the scope of the invention as long as the two therapies act concurrently to provide pain relief to the treated subject.

The compositions of the invention comprise both an N-type calcium channel blocking compound and a second pain-relieving compound such as those mentioned above. Typically, the compositions also include pharmaceutically acceptable excipients. Commonly, they are provided in the form of tablets, pills, troches, lozenges, suppositories, injectable solutions, controlled-release formulations, transdermal skin patches and the like, that provide a unit dosage of each active ingredient.

The foregoing compositions provide simultaneous delivery of an N-type calcium channel blocker and a second pain-relieving compound having a different mechanism of action. Since duration of action of two pain relieving compounds is often different, it is sometimes appropriate to use a different dosing schedule for each compound. Therefore, the methods of the invention also include methods wherein a subject is treated concurrently with such combinations of analgesic compounds, even though they may not be administered simultaneously.

The preferred route of administration of two concurrently administered analgesics may also differ. Therefore, the invention includes methods wherein the two analgesic compounds are administered by different routes. For example, one compound may be administered orally, and the other by a transdermal or transmucosal method.

Also within the scope of the invention, an N-type calcium channel blocker may be administered concurrently with the use of a non-pharmacological pain treatment such as transcutaneous electrical nerve stimulation (TENS), percutaneous electrical nerve stimulation (PENS), acupuncture, or a surgical intervention procedure.

### MODES OF CARRYING OUT THE INVENTION

Pain comes in many forms, and ways to manage it are essential to the health and comfort of affected individuals. Because pain takes many forms, treatments of pain also must take many forms so the treatment can be tailored to the needs of the patient. For example, treatments for acute pain need to work quickly, but it may be desirable for a drug treating acute pain to clear from the patient's body quickly so that adverse effects dissipate quickly. Chronic pain may require drugs with longer-lasting effects; these may not need to act as quickly, but because chronic pain demands protracted treatment, certain side-effects and interactions with other medications may be much more problematic in a treatment for chronic pain. Breakthrough pain, which overwhelms the effectiveness of a primary pain treatment protocol, may require a fast-acting treatment that can be administered to a patient already medicated with maximum amounts of a primary treatment. And extreme pain may justify usage of drugs with substantial adverse effects if no other satisfactory options exist; the opioids are an example of pain relievers with known potential for development of dependence that are nevertheless essential to treat severe pain.

Many patients benefit from a combination of pain treatment therapies, either because they have a combination of different types of pain or because no single treatment provides effective relief. Combinations offer certain obvious advantages where different types of pain are involved: each type of pain may be alleviated by one component of the combination. However, combinations also provide other advantages. Where the combination embodies two different modes of action, the effects may be additive while any side-effects may not. The overall effect of a combination may be one that could not be achieved with a safe dose of any single drug; for example, hepatotoxicity of acetaminophen limits the dosage that can be used. Using multiple drugs having a single mode of action may not be feasible; for example, the acidic NSAID compounds tend to cause stomach bleeding, and the effect depends on the total dose of such drugs administered, so mixing compounds within this family generally does not solve such problems. Furthermore, there may be unexpected problems from one mode of action: the COX-2 inhibitors are now being studied for a possible tendency to increase cardiac problems such as heart attack and stroke, and so far it is not clear whether the effect is related to the mechanism of action. If one mode of action has negative consequences, whether generally or for a particular subject in need of treatment, using combinations of drugs having the same mode of action could increase the risk of such side effects. Thus it is highly desirable to have new combination pain treatment methods and drug compositions that allow optimization of the pain-relieving characteristics, and it is also desirable to employ mixed modes of action in such combination pain therapies to avoid increasing the risk factors associated with any single mechanism of action or family of compounds.

The present compositions and methods are in one aspect intended for the treatment of chronic pain, including neuropathic pain such as diabetic peripheral neuropathy, post-herpetic neuralgia, trigeminal neuralgia, AIDS related neuropathy, cancer pain, inflammatory pain, osteoarthritis pain, rheumatoid arthritis pain, and fibromyalgia, and for the treatment of acute pain indications such as nociceptive pain and post-operative pain.

COX-1 refers to the constitutive cyclooxygenase involved in synthesis of prostaglandins that is widely dispersed in tissues, and is especially prevalent in the stomach, kidney, and in platelets in the blood. COX-2 is an inducible cyclooxygenase whose concentration increases where inflammation develops.

“Calcium channel blocker” as used herein refers to a compound that inhibits the transmission of calcium ions through a membrane, where that transmission is controlled by a protein referred to as a calcium ion channel.

The term “selective” as used to describe calcium channel blockers herein means that the compound is more effective, or is effective at a lower dosage, for one activity than for another similar activity. Thus a selective N-type calcium channel blocker is more effective at blocking N-type calcium channels than it is for blocking L-type or T-type calcium channels, for example.

The term “analgesic” as used herein is a general term referring to a compound or composition that has pain-relieving capability in at least some subjects in need of pain relief; when used as an adjective, it refers to the partial or complete remediation of pain.

The terms “alleviate”, “relieve” and “ameliorate” as used herein all include a reduction or elimination of the perception of pain as well as conversion of the unpleasant experience of pain into a more tolerable or less unpleasant experience.

“Peptidyl” as used herein refers to compounds that are N-acylated alpha-amino acids, and to alpha-amino acids generally; it also includes alpha-amino acids where the carboxylate group exists as an ester or amide. It does not require or suggest that the amino acid portion of the molecule is that of a naturally occurring amino acid, nor does it indicate or suggest that the amino acid portion has the stereochemical configuration associated with naturally occurring amino acids.

Thus the term “non-peptidyl” as used herein excludes compounds that are classically considered peptides, but it also excludes alpha-amino acids and their esters and amides.

The term “non-peptidyl N-type calcium channel blocker” thus refers to any compound having N-type calcium channel blocking activity, where the compound is not an alpha-amino acid or an ester or amide of an alpha-amino acid.

“Opioid” as used herein refers to any compound that binds at the opiate receptors, and includes natural as well as synthetic substances: it is a generic term that includes all compounds having morphine-like activity. Textbook of Pain, 4^{th} ed., P. D. Wall and R. Melzack, ed., Elsevier Science, pg. 1192 (2003).

“Non-steroidal anti-inflammatory drug” or “NSAID” as used herein refers to the non-opioid analgesics other than the selective COX-2 inhibitors, and excluding N-type calcium channel blockers. The terms therefore include several chemical families. Some NSAIDs inhibit both COX-1 and COX-2 at biologically-relevant concentrations. The term “NSAID” is used herein to encompass the classic anti-inflammatory drugs such as aspirin, ibuprofen, other salicylates, indomethacin, naproxen, diclofenac, piroxicam, mefenamic acid, and meclofenamic acid; it also encompasses acetaminophen, nabumetone, nemuselide, and meloxicam.

“Adjuvant analgesic” as used herein refers to compounds that are not primarily used as pain treatments, but that provide pain relief in at least some conditions. Textbook of Pain, 4^{th} ed., P. D. Wall and R. Melzack, ed., Elsevier Science, pp. 1496-1502 (2003). This category includes certain corticosteroids, neuroleptics, antihistamines, benzodiazepines, antidepressants, anticonvulsants, local anesthetics delivered orally, alpha-adrenergic agonists, NMDA antagonists, and bisphosphonates as well as miscellaneous compounds such as capsaicin, baclofen, gabapentin, calcitonin, pimozide, and scopolamine.

Where two compounds or treatments are described herein as being used or administered “concurrently”, that term means that the treatments are administered to a single subject, and that the timing of the administration of each treatment is such that both treatments provide at least some pain relief to the subject simultaneously. Thus the compounds or treatments may be administered at different times or by different means, but the time during which one treatment affords measurable pain relief overlaps with the time during which the other treatment affords measurable pain relief.

“Tetracyclic or pentacyclic channel blockers”: The compounds within U.S. Pat. Nos. 6,011,035; 6,294,533; 6,310,059; 6,387,897, and 6,617,322 and published U.S. Patent Applications No. 2003/0045530, US2003/0199523, US2004/0034035, US2004/0044004, US2004/0147529, US2004/0180323, US2004/0192703, US2004/0209872, US2004/0259866, US2004/0266784, and US2005/0014748 that have a 5-6 membered nitrogen-containing saturated central ring that is linked to a benzhydryl group or a partially or fully saturated benzhydryl group and is also linked to another group containing at least one additional ring are referred to herein as “tetracyclic or pentacyclic channel blockers”. Typically the central ring in these compounds is a piperidine or a piperazine, and often it is 1,4-disubstituted; the central ring is also sometimes a pyrrolidine. Often the benzhydryl or saturated or partially benzhydryl group, which may optionally be substituted, is linked to position 1 of the central ring.

“TENS” or transcutaneous electrical nerve stimulation and “PENS” or percutaneous electrical nerve stimulation as used herein refer to methods wherein electrical stimulation is used to dull or eliminate the experience of pain. TENS and PENS are described for example in Textbook of Pain, 4^{th} ed., P. D. Wall and R. Melzack, ed., Elsevier Science, pp. 1341-46 (2003).

“Benzhydryl” as used herein refers to a chemical group wherein two phenyl rings are attached to a single carbon atom, which is an sp³ carbon that is the atom through which the benzhydryl group is attached to the remainder of the molecule comprising it. A benzhydryl group may also have substituents on either or both phenyl groups and may have another group or substituent on the sp³ carbon in addition to the two rings.

A “saturated or partially saturated” benzhydryl as used herein refers to a benzhydryl group in which at least one of the phenyl rings is reduced to a saturated cyclohexane or partially reduced to a cyclohexene or cyclohexadiene. It also includes groups in which both phenyl rings have been so reduced or partially reduced.

The N-type calcium channel blockers useful for the present invention include non-peptidyl compounds that treat pain by blocking N-type calcium channels. In some embodiments, the N-type calcium channel blocker is a tetracyclic or pentacyclic channel blocker as described above, or it is a compound having an optionally substituted benzhydryl group linked to a nitrogen in a 1,3-disubstituted pyrrolidine, a 1,4-disubstituted piperidine or a 1,4-disubstituted piperazine. Often, it is a compound of formula (1):

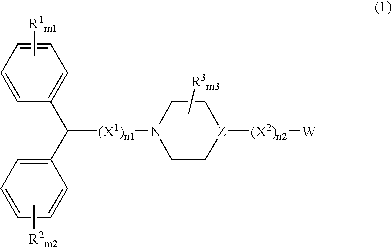

wherein each m¹ and m² is independently 0-5;

m³ is 0-2;

each of R¹, R² and R³ is independently a noninterfering substituent;

Z is N or CH;

wherein each of n¹ and n² is independently 0 or 1;

X¹ and X² are linkers; and

W is Ar or Cy, wherein
Ar represents one or two substituted or unsubstituted aromatic or heteroaromatic rings, and
Cy represents one or two substituted or unsubstituted aliphatic cyclic or heterocyclic moieties, or consists of one substituted or unsubstituted aliphatic cyclic or heterocyclic moiety and one substituted or unsubstituted aromatic or heteroaromatic moiety,
or a pharmaceutically acceptable salt of such a compound.

As used herein, the terms “alkyl,” “alkenyl” and “alkynyl” include straight-chain, branched-chain and cyclic monovalent substituents, containing only C and H when they are unsubstituted or unless otherwise noted. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (lower alkyl) or 2-6C (lower alkenyl or lower alkynyl).

Where an aryl, alkyl, alkenyl, or alkynyl group is described as “containing” one or more heteroatoms, the specified heteroatoms may replace any or all of the carbon and/or hydrogen atoms in the aryl, alkyl, alkenyl, or alkynyl group.

Additional examples of optionally substituted alkyl groups include propyl, tert-butyl, etc., and including cycloalkyls such as cyclopropyl, cyclobutyl, cyclopentyl, cyclohexyl, cycloheptyl, etc.; examples of optionally substituted alkenyl groups include allyl, crotyl, 2-pentenyl, 3-hexenyl, 2-cyclopentenyl, 2-cyclohexenyl, 2-cyclopentenylmethyl, 2-cyclohexenylmethyl, etc.; C₁₋₆ alkyl and alkenyl are preferred.

As used herein, “acyl” encompasses the definitions of alkyl, alkenyl, alkynyl, each of which is coupled to an additional residue through a carbonyl group.

“Aromatic” moiety or “aryl” moiety refers to a monocyclic or fused bicyclic moiety such as phenyl, furanyl, or naphthyl, and includes heteroaromatic (heteroaryl) rings; “heteroaromatic” specifically refers to monocyclic or fused bicyclic ring systems containing one or more heteroatoms selected from O, S and N. The inclusion of a heteroatom permits inclusion of 5-membered rings as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroarylalkyl” refer to aromatic and heteroaromatic systems which are coupled to another residue through a carbon chain, including substituted or unsubstituted, saturated or unsaturated, carbon chains, typically of 1-8C, or the hetero forms thereof. These carbon chains may also include a carbonyl group, thus making them able to provide substituents as an acyl or heteroacyl moiety.

In general, any alkyl, alkenyl, alkynyl, acyl, or aryl group contained in a substituent may itself optionally be substituted by additional substituents. The nature of these substituents is similar to those recited with regard to the primary substituents themselves. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments. However, alkyl substituted by aryl, amino, alkoxy, and the like would be included.

Examples of halogen include fluorine, chlorine, bromine, and iodine, with fluorine and chlorine preferred.

Examples of optionally substituted hydroxyl and thiol groups include optionally substituted alkyloxy or alkylthio (e.g., C₁₋₁₀ alkyl, such as methyl, ethyl, propyl, isopropyl, butyl, isobutyl, sec-butyl, tert-butyl, pentyl, cyclopropyl, cyclobutyl, cyclopentyl, cyclohexyl, cycloheptyl, etc.); an optionally substituted arylalkyloxy or arylalkylthio (e.g., phenyl-C₁₋₄ alkyl, e.g., benzyl, phenethyl, etc.). Where there are two adjacent hydroxyl or thiol substituents, the heteroatoms may be connected via an optionally substituted alkylene group such as O(CH₂)ₙO O—C(CH₃)₂—O and S(CH₂)ₙS (where n=1-5). Examples include methylenedioxy, ethylenedioxy, etc. Oxides of thio-ether groups such as sulfoxides and sulfones are also envisioned.

Examples of optionally substituted hydroxyl groups also include optionally substituted C₂₋₄alkanoyl (e.g., acetyl, propionyl, butyryl, isobutyryl, etc.), C₁₋₄ alkylsulfonyl (e.g., methanesulfonyl, ethanesulfonyl, etc.) and an optionally substituted aromatic and heterocyclic carbonyl group including benzoyl, pyridinecarbonyl, etc.

Substituents on optionally substituted amino groups may bind to each other to form a cyclic amino group (e.g., 5- to 6-membered cyclic amino, etc., such as tetrahydropyrrole, piperazine, piperidine, pyrrolidine, morpholine, thiomorpholine, pyrrole, imidazole, etc.). Said cyclic amino group may have a substituent, and examples of the substituents include halogen (e.g., fluorine, chlorine, bromine, iodine, etc.), nitro, cyano, hydroxy group, thiol group, amino group, carboxyl group, an optionally halogenated C₁₋₄ alkyl (e.g., trifluoromethyl, methyl, ethyl, etc.), an optionally halogenated C₁₋₄ alkoxy (e.g., methoxy, ethoxy, trifluoromethoxy, trifluoroethoxy, etc.), C₂₋₄ alkanoyl (e.g., acetyl, propionyl, etc.), C₁₋₄ alkylsulfonyl (e.g., methanesulfonyl, ethanesulfonyl, etc.) the number of preferred substituents is 1 to 3.

An amino group may also be substituted once or twice (to form a secondary or tertiary amine) with a group such as an optionally substituted alkyl group including C₁₋₁₀alkyl (e.g., methyl, ethyl propyl etc.); an optionally substituted alkenyl group such as allyl, crotyl, 2-pentenyl, 3-hexenyl, etc., or an optionally substituted cycloalkyl group such as cyclopropyl, cyclobutyl, cyclopentyl, cyclohexyl, cycloheptyl, etc. In these cases, C₁₋₆ alkyl, alkenyl and cycloalkyl are preferred. The amine group may also be optionally substituted with an aromatic or heterocyclic group, arylalkyl (e.g., phenylC₁₋₄alkyl) or heteroarylalkyl for example, phenyl, pyridine, phenylmethyl (benzyl), phenethyl, pyridinylmethyl, pyridinylethyl, etc. The heterocyclic group may be a 5 or 6 membered ring containing 1-4 heteroatoms.

An amino group may be substituted with an optionally substituted C₂₋₄ alkanoyl, e.g., acetyl, propionyl, butyryl, isobutyryl etc., or a C₁₋₄alkylsulfonyl (e.g., methanesulfonyl, ethanesulfonyl, etc.) or a carbonyl or sulfonyl substituted aromatic or heterocyclic ring, e.g., benzenesulfonyl, benzoyl, pyridinesulfonyl, pyridinecarbonyl etc. The heterocycles are as defined above.

Examples of optionally substituted carbonyl groups or sulfonyl groups include optionally substituted forms of such groups formed from various hydrocarbyls such as alkyl, alkenyl and 5- to 6-membered monocyclic aromatic group (e.g., phenyl, pyridyl, etc.), as defined above.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Examples of inorganic bases with alkali metal hydroxides (e.g., sodium hydroxide, potassium hydroxide, etc.), alkaline earth metal hydroxides (e.g., of calcium, magnesium, etc.), and hydroxides of aluminum, ammonium, etc. Examples of organic bases include trimethylamine, triethylamine, pyridine, picoline, ethanolamine, diethanolamine, triethanolamine, dicyclohexylamine, N,N′-dibenzylethylenediamine, etc. Examples of inorganic acids include hydrochloric acid, hydrobromic acid, nitric acid, sulfuric acid, phosphoric acid, etc. Examples of organic acids include formic acid, oxalic acid, acetic acid, tartaric acid, methanesulfonic acid, benzenesulfonic acid, malic acid, methanesulfonic acid, benzenesulfonic acid, p-toluenesulfonic acid, etc. Also included are salts with basic amino acids such as arginine, lysine, ornithine, etc., and salts with acidic amino acids such as aspartic acid, glutamic acid, etc.

Where a composition of the invention includes two or more active ingredients, it is also possible that the two are capable for forming a salt together; for example, a salicylic acid or acidic NSAID compound can form a salt with a piperazine-containing N-type calcium channel blocker. These salts that do not include an additional counterion are also within the scope of the invention.

In addition, in some cases, the compounds of the invention contain one or more chiral centers. The invention includes the isolated stereoisomeric forms as well as mixtures of stereoisomers in varying degrees of chiral purity, as well as compositions wherein one, two, or all of two or more active components are enantiomerically enriched.

The compounds of the invention may be in the form of a salt if appropriate or in the form of a prodrug. With respect to compounds of the invention that contain chiral centers (and each compound of the invention contains at least one chiral center) the compounds may be in the form of isolated stereoisomers or mixtures of various stereoisomers, including enantiomeric mixtures, equimolar mixtures of all possible stereoisomers, or various degrees of chiral or optical purity.

The linkers represented by X¹ and X² are alkylene or alkenylene moieties optionally including one or more hetero-atoms selected from N, O, and S and optionally substituted by one or more noninterfering substituents. The number of members in the chain in the linkers is 1-10, preferably 1-6. Where Z is N, it is often preferred that one of the nitrogen atoms in the piperazine ring is acylated and the other is not, so that only one of the nitrogen atoms is basic. Thus in such compounds, one of X¹ and X² is often an acyl group that is attached to the central ring through the carbonyl carbon atom.

Noninterfering substituents generally are optionally substituted alkyl (1-10C), alkenyl (2-10C), alkynyl (2-10C), aryl (5-12 ring members), arylalkyl (7-16C) or arylalkenyl (7-16C) each optionally having one or more C, generally 1-4C, replaced by heteroatoms (N, O and/or S) and wherein said optional substituents on alkyl, alkenyl, etc., may include one or more ═O. Thus substituents include embodiments wherein these substituents may form an acyl, amide, or ester linkage with the atom to which it is bound. The substituents include, as well, one or more halo, CF₃, CN, OCF₃, NO₂, NO, SO, SO₂, NR₂, OR, SR, COOR, and/or CONR₂, wherein R is H or optionally substituted alkyl, alkenyl, alkynyl, aryl, arylalkyl, or arylalkenyl, as described above, and wherein S may be oxidized, and wherein two substituents may form a 3-7 membered saturated or unsaturated ring, said ring optionally itself substituted and optionally containing one or more heteroatoms (N, S, O). When a substituent shown in a formula is mandatory, it may also be H.

Thus, in some embodiments, non-interfering substituents in general include, but are not limited to, alkyl, alkenyl, alkynyl, aryl, arylalkyl, acyl, ═O, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, wherein each R is independently H or alkyl (1-8C), CN, CF₃, and NO₂, and like substituents. In some preferred embodiments, the noninterfering substituents are selected from halo, C1-C4 alkyl, CF₃, C1-C4 alkoxy, CN, NR′₂, COOR, SO₂R′, SR′, and CONR′₂, wherein each R′ is H or C1-C4 alkyl.

In each case, preferably when X² is present, X² represents a linker which spaces the Ar or Cy moiety from Z at a distance of 3-20 Å, and may contain at least one heteroatom which is nitrogen or oxygen. Included in such linkers are amines and carbonyl functionalities, including amides. The linker may also be unsaturated or may be an alkylene group. Typically, X₂ is (CH₂)₁₋₈ or (CH₂)₁₋₅—CH═CH—(CH₂)₀₋₃—. Similarly, X¹, when present, spaces the benzhydryl moiety from the nitrogen of the heterocyclic ring at a distance of 3-20 Å and may contain a heteroatom. Preferred embodiments are similar to those for X².

In both cases, when there are two aromatic or heterocyclic moieties, X² must accommodate this and a typical embodiment is —(CH₂)₀₋₆—CH, which may also contain a π-bond.

Thus, in preferred forms of formulas (1a) and (1b), n¹ is 0, or n¹ is 1 and X¹ is (CH₂)₁₋₅CO(CH₂)₀₋₃, (CH₂)₁₋₅NH(CH₂)₀₋₃, (CH₂)₁₋₅CONH(CH₂)₀₋₃, or (CH₂)₁₋₅NHCO(CH₂)₀₋₃.

The preferred embodiments for X² are similar except that in instances where Ar or Cy represent two rings, the two rings are coupled to CH as the terminal portion of the linker X². When X¹ and X² are selected from these preferred embodiments, although it is preferred that 1¹ and 1² are both 0, substitution by R¹ and R² in the benzhydryl system is permitted as set forth in the description of the invention above, and may also include, in these instances, a para-fluoro substituent.

W is sometimes Ar where Ar is a single phenyl moiety that is optionally substituted, and sometimes it is preferable that Ar is two phenyl moieties located on the terminal carbon of X². Thus Ar and X² can form a benzhydryl group linked to the central ring. In some preferred embodiments, this benzhydryl group is unsubstituted, and it may be linked to the central ring through an acyl group. For example, X²—W may represent —C(O)—(CH₂)ₚ—CH(Ph)₂, and in preferred embodiments p is often 1 or 2.

It is believed that halogenation of the compounds of the invention is helpful in modulating the in vivo half-life, and it may be advantageous to include halogen substituents as R¹ and R². Thus in some preferred embodiments, one or both of R¹ and R² is halo, preferably F or Cl. In other embodiments, R¹ and R², if both present, are independently chosen from C1-C4 alkyl, CN, CF₃ and C1-C4 alkoxy, or one of R¹ and R² is halo and the other is chosen from C1-C4 alkyl, CN, CF₃ and C1-C4 alkoxy. Often it is preferable that at least one of m¹ and m² is 0. In formula (1), such substituents may be included on Ar and/or Cy. Substituents for Ar are similar to those optionally present as R¹ and R². Ar may optionally be substituted by up to 5 such substituents, but it is often preferred that Ar contain 1 substituent or 0-2 such substituents.

The N-type calcium channel blocking compounds may also be supplied as pharmaceutically acceptable salts, as they often include a basic amine group. Pharmaceutically acceptable salts include the acid addition salts which can be formed from inorganic acids such as hydrochloric, sulfuric, and phosphoric acid or from organic acids such as acetic, citric, tartaric, propionic, glutamic, glutaric, glucuronic, alkylsulfonic, and arylsulfonic acids as well as acid ion-exchange resins.

In some embodiments it is preferred that one of n¹ and n² is 1, or that both n¹ and n² are 1. In some embodiments, W is Ar, and Ar represents an optionally substituted phenyl moiety. Z is often N, or Z may be CH. Sometimes m¹ and m² are both 0; in other embodiments each of m¹ and m² is 1, and R¹ and R² are halogen substituents. In other embodiments, m¹ and m² may be different, and R¹ and R² when present may be independently selected from alkyl, alkoxy, F, Cl, CN, CF₃, and NO₂. R³, when present, is sometimes alkyl and sometimes ═O. When m³ is 1, R³ is often ═O (carbonyl), and when m³ is 2, R³ is often alkyl, preferably methyl.

In some embodiments, then, the N-type calcium channel blockers used for the invention comprise a 1,4-disubstituted piperidine or 1,4-disubstituted piperazine ring, wherein the substituent at position 1 (the nitrogen atom in the piperidine ring, or one of the two nitrogen atoms in the piperazine ring) comprises a benzhydryl group which may be connected to N through a linker. Sometimes the N-type calcium channel blocker is 1-(4-benzhydrylpiperazin-1-yl)-3,3-diphenylpropan-1-one; 6,6-diphenyl-1-[4-(3-phenyl-2-propen-1-yl)-piperazin-1-yl]hexan-1-one; 6,6-diphenyl-1-[4-(cyclohexylmethylamino)-piperidin-1-yl]hexan-1-one; 6,6-bis-(4-fluorophenyl)-1-[4-(3,4,5-trimethoxyphenylmethyl)piperazin-1-yl]hexane, or 4-benzhydryl-piperazine-1-carboxylic acid benzhydryl-amide. In some preferred embodiments, the N-type calcium channel blocker is 1-(4-benzhydrylpiperazin-1-yl)-3,3-diphenylpropan-1-one. Salts of these compounds are equally useful as long as the counterion is pharmaceutically acceptable.

Also suitable as the N-type calcium channel blocker are the following compounds:
6,6-Bis-(4-fluoro-phenyl)-1-[4-(2-phenylsulfanyl-ethyl)-piperazin-1-yl]-hexan-1-one;
1-[6,6-Bis-(4-fluoro-phenyl)-hexyl]-4-[2-(4-fluoro-phenoxy)-ethyl]-piperazine;
1-{4-[2-(Benzo[1,3]dioxol-5-yloxy)-ethyl]-piperazin-1-yl}-6,6-bis-(4-fluoro-phenyl)-hexan-1-one;
1-[6,6-Bis-(4-fluoro-phenyl)-hexyl]-4-(2-phenylsulfanyl-ethyl)-piperazine;
1-[6,6-Bis-(4-fluoro-phenyl)-hexyl]-4-[2-(4-methoxy-phenoxy)-ethyl]-piperazine;
1-{4-[2-(2,4-Difluoro-phenoxy)-ethyl]-piperazin-1-yl}-6,6-bis-(4-fluoro-phenyl)-hexan-1-one;
6,6-Bis-(4-fluoro-phenyl)-1-[4-(2-phenoxy-ethyl)-piperazin-1-yl]-hexan-1-one;
1-{4-[2-(2,4-Dichloro-phenoxy)-ethyl]-piperazin-1-yl}-6,6-bis-(4-fluoro-phenyl)-hexan-1-one;
6,6-Bis-(4-fluoro-phenyl)-1-{4-[2-(4-methoxy-phenoxy)-ethyl]-piperazin-1-yl}-hexan-1-one;
1-[6,6-Bis-(4-fluoro-phenyl)-hexyl]-4-(2-phenoxy-ethyl)-piperazine;
6,6-Bis-(4-fluoro-phenyl)-1-{4-[2-(3,4,5-trimethoxy-phenoxy)-ethyl]-piperazin-1-yl}-hexan-1-one;
1-{4-[2-(Benzothiazol-2-ylsulfanyl)-ethyl]-piperazin-1-yl}-6,6-bis-(4-fluoro-phenyl)-hexan-1-one;
[4-(2-{4-[6,6-Bis-(4-fluoro-phenyl)-hexanoyl]-piperazin-1-yl}-ethoxy)-2,3,6-trimethyl-phenyl]-carbamic acid tert-butyl ester;
4-(2-{4-[6,6-Bis-(4-fluoro-phenyl)-hexyl]-piperazin-1-yl}-ethoxy)-2,3,6-trimethyl-phenylamine;
1-[6,6-Bis-(4-fluoro-phenyl)-hexyl]-4-[2-(2,4-dichloro-phenoxy)-ethyl]-piperazine;
[2-(4-{4-[6,6-Bis-(4-fluoro-phenyl)-hexyl]-piperazin-1-ylmethyl}-2,6-di-tert-butyl-phenoxy)-ethyl]-dimethyl-amine;
4-{4-[6,6-Bis-(4-fluoro-phenyl)-hexyl]-piperazin-1-ylmethyl}-2,6-di-tert-butyl-phenol;
1-[4-(3,5-Di-tert-butyl-4-methoxy-benzoyl)-piperazin-1-yl]-6,6-bis-(4-fluoro-phenyl)-hexan-1-one;
1-[4-(3,5-Di-tert-butyl-4-methoxy-benzyl)-piperazin-1-yl]-6,6-bis-(4-fluoro-phenyl)-hexan-1-one;
1-[6,6-Bis-(4-fluoro-phenyl)-hexyl]-4-(3,5-di-tert-butyl-4-methoxy-benzyl)-piperazine;
{4-[6,6-Bis-(4-fluoro-phenyl)-hexyl]-piperazin-1-yl}-(3,5-di-tert-butyl-4-methoxy-phenyl)-methanone;
1-{4-[3,5-Di-tert-butyl-4-(2-dimethylamino-ethoxy)-benzoyl]-piperazin-1-yl}-6,6-bis-(4-fluoro-phenyl)-hexan-1-one;
1-Benzo[1,3]dioxol-5-ylmethyl-4-[6,6-bis-(4-fluoro-phenyl)-hexyl]-piperazine;
1-[6,6-Bis-(4-fluoro-phenyl)-hexyl]-4-(3,5-di-tert-butyl-benzyl)-piperazine;
{4-[6,6-Bis-(4-fluoro-phenyl)-hexyl]-piperazin-1-yl}-(3,5-di-tert-butyl-4-hydroxy-phenyl)-methanone;
1-[4-(3,5-Di-tert-butyl-4-hydroxy-benzyl)-piperazin-1-yl]-6,6-bis-(4-fluoro-phenyl)-hexan-1-one;
1-[4-(3,5-Dibromo-4-hydroxy-benzoyl)-piperazin-1-yl]-6,6-bis-(4-fluoro-phenyl)-hexan-1-one;
1-[4-(3,5-Di-tert-butyl-4-hydroxy-benzoyl)-piperazin-1-yl]-6,6-bis-(4-fluoro-phenyl)-hexan-1-one;
1-[4-(3,5-Di-tert-butyl-benzoyl)-piperazin-1-yl]-6,6-bis-(4-fluoro-phenyl)-hexan-1-one;
1-[6,6-Bis-(4-fluoro-phenyl)-hexyl]-4-(4-tert-butyl-benzyl)-piperazine;
1-[6,6-Bis-(4-fluoro-phenyl)-hexyl]-4-(9H-thioxanthen-9-yl)-piperazine;
2-{4-[6,6-Bis-(4-fluoro-phenyl)-hexyl]-piperazin-1-yl}-benzothiazole;
6,6-Bis-(4-fluoro-phenyl)-1-(4-pyrimidin-2-yl-piperazin-1-yl)-hexan-1-one;
2-{4-[6,6-Bis-(4-fluoro-phenyl)-hexyl]-piperazin-1-yl}-pyrimidine;
6,6-Bis-(4-fluoro-phenyl)-1-[4-(9H-thioxanthen-9-yl)-piperazin-1-yl]-hexan-1-one;
1-[6,6-Bis-(4-fluoro-phenyl)-hexyl]-4-(3,4,5-trimethoxy-benzyl)-piperazine-2-carboxylic acid ethyl ester;
6,6-Bis-(4-fluoro-phenyl)-1-{4-[2-(3,4,5-trimethoxy-benzylamino)-ethyl]-piperazin-1-yl}-hexan-1-one;
9,9-Bis-(4-fluoro-phenyl)-1-[4-(3,4,5-trimethoxy-benzyl)-piperazin-1-yl]-nonan-1-one;
(2-{4-[6,6-Bis-(4-fluoro-phenyl)-hexyl]-piperazin-1-yl}-ethyl)-phenyl-amine;
1-[9,9-Bis-(4-fluoro-phenyl)-nonyl]-4-(3,4,5-trimethoxy-benzyl)-piperazine;
(4-{4-[Bis-(4-fluoro-phenyl)-methoxy]-butyl}-piperazin-1-yl)-(3,4,5-trimethoxyphenyl)-methanone;
6,6-Bis-(4-fluoro-phenyl)-1-[4-(4-trifluoromethoxy-benzoyl)-piperazin-1-yl]-hexan-2-one;
1-[4-(4-Bromo-benzoyl)-piperazin-1-yl]-6,6-bis-(4-fluoro-phenyl)-hexan-1-one;
6,6-Bis-(4-fluoro-phenyl)-5-hydroxy-1-[4-(3,4,5-trimethoxy-benzoyl)-piperazin-1-yl]-hexan-1-one;
1-{4-[Bis-(4-fluoro-phenyl)-methoxy]-butyl}-4-(3,4,5-trimethoxy-benzyl)-piperazine;
6,6-Bis-(4-fluoro-phenyl)-6-hydroxy-1-[4-(3,4,5-trimethoxy-benzoyl)-piperazin-1-yl]-hexan-1-one;
4-[6,6-Bis-(4-fluoro-phenyl)-hexyl]-1-(3,4,5-trimethoxy-benzyl)-piperazine-2-carboxylic acid;
4-[6,6-Bis-(4-fluoro-phenyl)-hexanoyl]-1-(3,4,5-trimethoxy-benzyl)-piperazin-2-one;
1-[6,6-Bis-(4-fluoro-phenyl)-hexanoyl]-4-(3,5-di-tert-butyl-4-methoxy-benzoyl)-piperazin-2-one;
1-[6,6-Bis-(4-fluoro-phenyl)-hexyl]-4-(3,4,5-trimethoxy-benzoyl)-piperazin-2-one;
4-[6,6-Bis-(4-fluoro-phenyl)-hexyl]-1-(3,4,5-trimethoxy-benzyl)-piperazin-2-one;
4-[6,6-Bis-(4-fluoro-phenyl)-hexanoyl]-1-(3,5-di-tert-butyl-4-methoxy-benzyl)-piperazin-2-one;
4-[6,6-Bis-(4-fluoro-phenyl)-hexanoyl]-1-[2-(4-fluoro-phenoxy)-ethyl]-piperazin-2-one;
1-[6,6-Bis-(4-fluoro-phenyl)-hexyl]-4-(3,5-di-tert-butyl-4-methoxy-benzoyl)-piperazin-2-one;
4-[6,6-Bis-(4-fluoro-phenyl)-hexanoyl]-1-(3,5-di-tert-butyl-4-methoxy-benzoyl)-piperazin-2-one;
1-[6,6-Bis-(4-fluoro-phenyl)-hexyl]-4-(3,5-di-tert-butyl-4-methoxy-benzyl)-piperazin-2-one;
1-[6,6-Bis-(4-fluoro-phenyl)-hexanoyl]-4-(3,5-di-tert-butyl-4-hydroxy-benzoyl)-piperazin-2-one;
6,6-Bis-(4-fluoro-phenyl)-1-[4-(3,4,5-trimethoxy-benzoyl)-piperazin-1-yl]-hex-5-en-1-one;
1-{4-[2-(3,4-Dimethoxy-phenoxy)-ethyl]-piperazin-1-yl}-6,6-bis-(4-fluoro-phenyl)-hexan-1-one;
1-[6,6-Bis-(4-fluoro-phenyl)-hexyl]-4-[2-(3,4-dimethoxy-phenoxy)-ethyl]-piperazine;
1-[6,6-Bis-(4-fluoro-phenyl)-hexyl]-4-(3,4,5-trimethoxy-benzyl)-piperazine-2-carboxylic acid;
4-[6,6-Bis-(4-fluoro-phenyl)-hexyl]-1-(3,4,5-trimethoxy-benzyl)-piperazine-2-carboxylic acid ethyl ester;
1-[6,6-Bis-(4-fluoro-phenyl)-hexyl]-4-(3,4-dimethoxy-benzyl)-piperazine;
1-[4-(3,4-Dimethoxy-benzoyl)-piperazin-1-yl]-6,6-bis-(4-fluoro-phenyl)-hexan-1-one;
1-[2-(Benzo[1,3]dioxol-5-yloxy)-ethyl]-4-[6,6-bis-(4-fluoro-phenyl)-hexyl]-piperazine;
1-[6,6-Bis-(4-fluoro-phenyl)-hexyl]-4-[2-(3,4,5-trimethoxy-phenoxy)-ethyl]-piperazine;
1-(4-Amino-2,3,5-trimethyl-phenoxy)-3-{4-[6,6-bis-(4-fluoro-phenyl)-hexyl]-piperazin-1-yl}-propan-2-ol;
1-{4-[3-(4-Amino-2,3,5-trimethyl-phenoxy)-2-hydroxy-propyl]-piperazin-1-yl}-6,6-bis-(4-fluoro-phenyl)-hexan-1-one;
1-(4-Benzothiazol-2-yl-piperazin-1-yl)-6,6-bis-(4-fluoro-phenyl)-hexan-1-one;
1-[6,6-Bis-(4-fluoro-phenyl)-hexyl]-4-(3,5-bis-trifluoromethyl-benzyl)-piperazine;
1-[4-(3,5-Bis-trifluoromethyl-benzoyl)-piperazin-1-yl]-6,6-bis-(4-fluoro-phenyl)-hexan-1-one;
1-[4-(4-tert-Butyl-benzoyl)-piperazin-1-yl]-6,6-bis-(4-fluoro-phenyl)-hexan-1-one;
1-[6,6-Bis-(4-fluoro-phenyl)-hexyl]-4-(4-bromo-benzyl)-piperazine;
2-(2-{4-[6,6-Bis-(4-fluoro-phenyl)-hexyl]-piperazin-1-yl}-ethylsulfanyl)-benzothiazole;
6,6-Bis-(4-fluoro-phenyl)-1-[4-(4-hydroxy-3,5-dimethoxy-benzoyl)-piperazin-1-yl]-hexan-1-one;
4-{4-[6,6-Bis-(4-fluoro-phenyl)-hexyl]-piperazin-1-ylmethyl}-2,6-dibromo-phenol;
1-[6,6-Bis-(4-fluoro-phenyl)-hexyl]-4-(4-trifluoromethoxy-benzyl)-piperazine;
(2-{4-[6,6-Bis-(4-fluoro-phenyl)-hexyl]-piperazin-1-yl}-ethyl)-(3,4,5-trimethoxy-benzyl)-amine;
1-{4-[2-(4-Fluoro-phenoxy)-ethyl]-piperazin-1-yl}-6,6-bis-(4-fluoro-phenyl)-hexan-1-one;
6,6-Bis-(4-fluoro-phenyl)-1-[4-(2-phenylamino-ethyl)-piperazin-1-yl]-hexan-1-one;
1-[6,6-Bis-(4-fluoro-phenyl)-hexyl]-4-[2-(2,4-difluoro-phenoxy)-ethyl]-piperazine;
N-(2-{4-[6,6-Bis-(4-fluoro-phenyl)-hexyl]-piperazin-1-yl}-2-oxo-ethyl)-benzamide;
N-(2-{4-[6,6-Bis-(4-fluoro-phenyl)-hexyl]-piperazin-1-yl}-2-oxo-ethyl)-4-chloro-benzamide;
N-(2-{4-[6,6-Bis-(4-fluoro-phenyl)-hexyl]-piperazin-1-yl}-2-oxo-ethyl)-4-methyl-benzamide;
N-(2-{4-[6,6-Bis-(4-fluoro-phenyl)-hexyl]-piperazin-1-yl}-2-oxo-ethyl)-4-isopropyl-benzamide;
N-(2-{4-[6,6-Bis-(4-fluoro-phenyl)-hexyl]-piperazin-1-yl}-2-oxo-ethyl)-4-tert-butyl-benzamide;
N-(2-{4-[6,6-Bis-(4-fluoro-phenyl)-hexyl]-piperazin-1-yl}-2-oxo-ethyl)-4-fluoro-benzamide;
N-(2-{4-[6,6-Bis-(4-fluoro-phenyl)-hexanoyl]-piperazin-1-yl}-2-oxo-ethyl)-benzamide;
N-(2-{4-[6,6-Bis-(4-fluoro-phenyl)-hexanoyl]-piperazin-1-yl}-2-oxo-ethyl)-4-chloro-benzamide;
N-(2-{4-[6,6-Bis-(4-fluoro-phenyl)-hexanoyl]-piperazin-1-yl}-2-oxo-ethyl)-4-methyl-benzamide;
N-(2-{4-[6,6-Bis-(4-fluoro-phenyl)-hexanoyl]-piperazin-1-yl}-2-oxo-ethyl)-4-isopropyl-benzamide;
N-(2-{4-[6,6-Bis-(4-fluoro-phenyl)-hexanoyl]-piperazin-1-yl}-2-oxo-ethyl)-4-tert-butyl-benzamide;
N-(2-{4-[6,6-Bis-(4-fluoro-phenyl)-hexanoyl]-piperazin-1-yl}-2-oxo-ethyl)-4-fluoro-benzamide;
1-[4-(2-Benzylamino-ethyl)-piperazin-1-yl]-6,6-bis-(4-fluoro-phenyl)-hexan-1-one;
1-{4-[2-(4-Chloro-benzylamino)-ethyl]-piperazin-1-yl}-6,6-bis-(4-fluoro-phenyl)-hexan-1-one;
6,6-Bis-(4-fluoro-phenyl)-1-{4-[2-(4-methyl-benzylamino)-ethyl]-piperazin-1-yl}-hexan-1-one;
6,6-Bis-(4-fluoro-phenyl)-1-{4-[2-(4-isopropyl-benzylamino)-ethyl]-piperazin-1-yl}-hexan-1-one;
1-{4-[2-(4-tert-Butyl-benzylamino)-ethyl]-piperazin-1-yl}-6,6-bis-(4-fluoro-phenyl)-hexan-1-one;
1-{4-[2-(4-Fluoro-benzylamino)-ethyl]-piperazin-1-yl}-6,6-bis-(4-fluoro-phenyl)-hexan-1-one;
6,6-Bis-(4-fluoro-phenyl)-1-[4-(1-hydroxy-pyridine-4-carbonyl)-piperazin-1-yl]-hexan-1-one;
4-{4-[6,6-Bis-(4-fluoro-phenyl)-hexyl]-piperazin-1-ylmethyl}-2,6-dimethoxy-phenol;
9,9-Diphenyl-1-[4-(3,4,5-trimethoxy-benzyl)-piperazin-1-yl]-nonan-1-one;
1-[4-(3,5-Di-tert-butyl-4-methoxy-benzoyl)-2-methyl-piperazin-1-yl]-6,6-bis-(4-fluoro-phenyl)-hexan-1-one;
1-[4-(3,5-Di-tert-butyl-4-methoxy-benzoyl)-3-methyl-piperazin-1-yl]-6,6-bis-(4-fluoro-phenyl)-hexan-1-one;
1 [4-(3,5-Di-tert-butyl-benzoyl)-2-methyl-piperazin-1-yl]-6,6-bis-(4-fluoro-phenyl)-hexan-1-one; and
1-[4-(4-tert-Butyl-benzoyl)-2-methyl-piperazin-1-yl]-6,6-bis-(4-fluoro-phenyl)-hexan-1-one. Salts of these compounds are also useful as long as the counterion is pharmaceutically acceptable.

Still further compounds suitable for use include:
1-{4-[4-(4-Fluoro-benzyl)-phenyl]-piperazin-1-yl}-3,3-diphenyl-propan-1-one;
1-[4-(3,5-Di-tert-butyl-4-hydroxy-benzyl)-piperazin-1-yl]-3,3-diphenyl-propan-1-one;
1-{4-[3-(4-Amino-2,3,5-trimethyl-phenoxy)-2-hydroxy-propyl]-piperazin-1-yl}-3,3-diphenyl-propan-1-one;
1-(4-Adamantan-1-ylmethyl-piperazin-1-yl)-3,3-diphenyl-propan-1-one;
1-(4-Benzothiazol-2-yl-piperazin-1-yl)-3,3-diphenyl-propan-1-one;
3,3-Diphenyl-1-{4-[2-(3,4,5-trimethoxy-phenoxy)-ethyl]-piperazin-1-yl}-propan-1-one;
1-{4-[2-(3,4-Dimethoxy-phenoxy)-ethyl]-piperazin-1-yl}-3,3-diphenyl-propan-1-one;
1-{4-[2-(Benzothiazol-2-ylsulfanyl)-ethyl]-piperazin-1-yl}-3,3-diphenyl-propan-1-one;
N-(2,6-Dimethyl-phenyl)-2-[4-(3,3-diphenyl-propionyl)-piperazin-1-yl]-acetamide;
2-[4-(3,3-Diphenyl-propionyl)-piperazin-1-yl]-N-methyl-N-phenyl-acetamide;
3,3-Diphenyl-1-[4-(1-phenyl-ethyl)-piperazin-1-yl]-propan-1-one;
1-[4-(2-Diallylamino-ethyl)-piperazin-1-yl]-3,3-diphenyl-propan-1-one;
1-[4-(2-Dipropylamino-ethyl)-piperazin-1-yl]-3,3-diphenyl-propan-1-one;
1-(4-sec-Butyl-piperazin-1-yl)-3,3-diphenyl-propan-1-one;
1-[4-(1-Ethyl-propyl)-piperazin-1-yl]-3,3-diphenyl-propan-1-one;
1-[4-(1-Methyl-piperidin-3-ylmethyl)-piperazin-1-yl]-3,3-diphenyl-propan-1-one;
1-(4-Heptyl-piperazin-1-yl)-3,3-diphenyl-propan-1-one;
3,3-Diphenyl-1-(4-pyridin-4-ylmethyl-piperazin-1-yl)-propan-1-one;
1-[4-(3,5-Dichloro-phenyl)-piperazin-1-yl]-3,3-diphenyl-propan-1-one;
1-(4-Cycloheptyl-piperazin-1-yl)-3,3-diphenyl-propan-1-one;
1-[4-(3,4-Dimethyl-phenyl)-piperazin-1-yl]-3,3-diphenyl-propan-1-one;
1-(4-Biphenyl-4-yl-piperazin-1-yl)-3,3-diphenyl-propan-1-one;
1-[4-(2,3-Dichloro-phenyl)-piperazin-1-yl]-3,3-diphenyl-propan-1-one;
1-[4-(1-Methyl-piperidin-4-ylmethyl)-piperazin-1-yl]-3,3-diphenyl-propan-1-one;
N-{2-[4-(3,3-Diphenyl-propionyl)-piperazin-1-yl]-ethyl}-3,4,5-trimethoxy-benzamide;
1-(4-Isopropyl-piperazin-1-yl)-3,3-diphenyl-propan-1-one;
1-[4-(3-Dimethylamino-propyl)-piperazin-1-yl]-3,3-diphenyl-propan-1-one;
3,3-Diphenyl-1-[4-(4-trimethylsilanyl-phenyl)-piperazin-1-yl]-propan-1-one;
3,3-Diphenyl-1-[4-(2-phenylamino-ethyl)-piperazin-1-yl]-propan-1-one;
1-{4-[2-(2,4-Difluoro-phenoxy)-ethyl]-piperazin-1-yl}-3,3-diphenyl-propan-1-one;
3,3-Diphenyl-1-{4-[2-(2,3,5,6-tetrafluoro-phenoxy)-ethyl]-piperazin-1-yl}-propan-1-one;
1-[4-(1-Benzyl-1H-benzoimidazol-2-yl)-piperazin-1-yl]-3,3-diphenyl-propan-1-one;
3,3-Diphenyl-1-[4-(phenyl-thiophen-2-yl-methyl)-piperazin-1-yl]-propan-1-one;
1-{4-[Cyclopropyl-(4-fluoro-phenyl)-methyl]-piperazin-1-yl}-3,3-diphenyl-propan-1-one;
(4-{2-[4-(3,3-Diphenyl-propionyl)-piperazin-1-yl]-ethoxy}-2,3,6-trimethyl-phenyl)-carbamic acid tert-butyl ester;
1-{4-[2-(4-Amino-2,3,5-trimethyl-phenoxy)-ethyl]-piperazin-1-yl}-3,3-diphenyl-propan-1-one;
1-{4-[2-(4-Methoxy-phenoxy)-ethyl]-piperazin-1-yl}-3,3-diphenyl-propan-1-one;
1-{4-[2-(Benzo[1,3]dioxol-5-yloxy)-ethyl]-piperazin-1-yl}-3,3-diphenyl-propan-1-one;
1-{4-[2-(2,4-Dichloro-phenoxy)-ethyl]-piperazin-1-yl}-3,3-diphenyl-propan-1-one;
1-{4-[2-(4-Fluoro-phenoxy)-ethyl]-piperazin-1-yl}-3,3-diphenyl-propan-1-one; and
3,3-Diphenyl-1-[4-(2-phenylsulfanyl-ethyl)-piperazin-1-yl]-propan-1-one; and salts thereof.

For use as treatment of animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa.

In general, for use in treatment, the compounds of formula (1) may be used individually or as mixtures of two or more compounds of formula (1) having N-type calcium channel activity. Where such a compound is used in combination with a second therapeutic compound having a different mechanism of action, the invention also includes its use with a mixture of such second therapeutic compounds. Where two or more of such compounds are used, the second therapeutic compounds may be from a single category of analgesics (e.g., NSAIDs, opioids, or adjuvant analgesics), or they may include two or more different analgesic categories. Depending on the mode of administration, the compounds will be formulated into suitable compositions to permit facile delivery, whether they are formulated as a mixture or separately.

The formulations, dosages, and routes of administration that are applicable to combinations of an N-type calcium channel blocker and a second analgesic are equally applicable to each individual active ingredient for use in methods where the two active compounds are administered as separate compositions.

It is sometimes preferable to combine an N-type calcium channel blocking compound with a second analgesic compound that operates by a different mechanism, where both are administered by the same route and on the same schedule. In such cases, the two compounds can optionally be combined in a single formulation and administered simultaneously as one composition. Where the methods of the invention comprise administering two or more pain relieving compounds, the compounds may be administered simultaneously in a single composition, or they may be administered essentially simultaneously as separate pills, for example, that are taken together. Alternatively, the compositions may be administered on different schedules or by different routes, or both. Thus, for example, an opioid such as fentanyl may be administered by a transdermal patch, while an N-type calcium channel blocking compound is separately administered as a tablet or pill that is administered orally: such combinations are within the scope of the invention where both compounds act concurrently to provide pain relief.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

Injection methods are sometimes appropriate routes for administration of pain relieving compositions of the invention, especially where rapid pain relief is required. These include methods for intravenous, intramuscular, subcutaneous, and other methods for internal delivery that bypass the mucosal and dermal barriers to deliver the composition directly into the subject's living tissues.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677. The present compositions can be utilized in such controlled-release delivery systems where appropriate.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, and the like as in understood in the art. Selection of a particular route for a given subject is well within the ordinary level of skill in the art. For example, rectal delivery as a suppository is often appropriate where the subject experiences nausea and vomiting that precludes effective oral delivery. Transdermal patches are commonly capable of delivering a controlled-release dosage over several days, and are thus suitable for subjects where this is appropriate. However, patches generally provide slow onset of pain relief, and may thus not be ideal for treatment of acute or breakthrough pain, for example.

Transmucosal delivery is also appropriate for some of the compositions and methods of the invention; for example, buccal delivery of fentanyl is known as a method to treat acute pain. Thus the compositions of the invention may be administered transmucosally using technology that is known for the delivery of NSAID, opioid, and adjuvant analgesics, as, long as the physical properties of the N-type calcium channel blocking compound are consistent with that mode of delivery.

For administration to animal or human subjects, the dosage of the N-type calcium channel blocking compounds of the invention is typically 10-2400 mg. However, dosage levels are highly dependent on the nature of the condition, the condition of the patient, the judgment of the practitioner, and the frequency and mode of administration as well as the nature and dosage of the other pain therapy administered to the subject. Selection of the dosage of such N-type calcium channel blocking compounds is within the skill of an ordinary artisan, and may be accomplished by starting at a relatively low dosage and increasing the dosage until an acceptable level of pain relief is provided.

Selection of dosages for the therapeutic compound that is not an N-type calcium channel blocker is readily done by those skilled in the art, using available information about the appropriate dosage of the compound or compounds to be administered. Thus a safe and effective dosage of an NSAID or a selective COX-2 inhibitor or of an opioid or an adjuvant analgesic can readily be established from known sources such as the FDA label for the particular compound. The dosage to be used in the combinations of the invention are similar to those that are appropriate for use without the N-type calcium channel blocker, but is often reduced to take advantage of the pain-relieving contribution provided by the N-type calcium channel blocker.

Sometimes, the dosage of a pain-relieving composition is determined empirically using patient-controlled analgesia (PCA). In this method, the patient is allowed to control the frequency with which small quantities of the pain relieving composition are administered. PCA not only allows determination of the dosage that is necessary to alleviate pain to an acceptable degree for the specific individual, it also often provides psychological relief by giving the treated subject a sense of control. Thus PCA is sometimes a suitable method for individually tailoring the dosage for the compositions and methods of the invention to the needs of a particular subject.

Administering the N-type calcium channel blocking compound in combination with an analgesic having a different mechanism of action generally provides an additive analgesic effect. Thus identification of subjects who would benefit from the compositions and methods of the present invention is also well within the ordinary skill in the arts: those subjects who require pain treatment are suitable subjects, and subjects who experience pain that is not well controlled by other methods are especially suited to the use of the methods and compositions of the present invention. Further, those subjects who require ongoing treatments for pain, chronic pain sufferers, are especially suitable subjects for the compositions and methods of the present invention; they will benefit from the mixture of mechanism of action that will reduce the tendency to develop tolerance for or dependence on the non-N-type calcium channel blockers and from the reduced risk of side effects from long-term use of a single drug.

Non-steroidal anti-inflammatory drugs, or NSAIDs, include compounds that are considered to inhibit both COX-1 and COX-2. NSAIDs that are suitable for use in the present invention include salicylates such as aspirin (acetylsalicylic acid), choline magnesium trisalicylate, diflunisal, magnesium salicylate, salsalate, and sodium salicylate. They also include acetaminophen, sulindac, diclofenac salts, fenoprofen salts, ibuprofen, indomethacin, ketoprofen, ketorolac tromethamine, meclofenamate salts, mefenamic acid, naproxen, carprofen, diflunisal, and etodolac. Selection of an appropriate dosage of each of these, including frequency of administration, is within the ordinary skill, and may be based on the body weight of the subject to be treated and known safety limits, for example.

NSAID drugs are typically delivered orally: because they tend to cause localized tissue damage if injected, among the NSAID pain treatments, only ketorolac tromethamine is often administered parenterally. NSAIDs may also be administered as suppositories. Frequently these routes are indicated where the subject is experiencing nausea or vomiting. Thus compositions of the invention that comprise an NSAID are also generally to be administered orally or as suppositories rather than by injection, and the selection of the route of administration is within the skill of the ordinary practitioner.

A subject who fails to respond to one NSAID compound or composition may nevertheless respond to a different NSAID compound or composition, as is well known in the art, and NSAID treatments are often suggested as a primary treatment for pain. Thus compositions and methods within the invention that comprise an NSAID compound are often appropriate first treatments for a given subject. If one composition or method of the invention that comprises an NSAID is inadequate for a particular subject, it is usually appropriate to next try another NSAID-containing composition or method of the invention before proceeding to methods or compositions comprising other types of analgesics. Indeed, it is often appropriate to include an NSAID with an opioid pain relief composition or method, since the NSAID may reduce the dosage of the opioid that is needed. The invention therefore includes methods and compositions wherein both an NSAID and an opioid, for example, are used in combination with an N-type calcium channel blocking compound.

Some COX-2 inhibitors suitable for use in the present invention include celecoxib (Celebrex®), rofecoxib (Vioxx®), valdecoxib (Bextra®), and lumaricoxib (Prexige®). As with NSAIDs, selection of dosages, frequency, and routes of administration for the compositions and methods comprising selective COX-2 inhibitors are within the skill of the ordinary practitioner.

Opioids are primarily indicated for subjects experiencing moderate to severe pain. Their long-term use can lead to tolerance, where a larger dose is required to achieve the same result that was earlier achieved by a lower dose. It can also lead to physical and/or psychological dependence. Some opioids suitable for use in the present invention include those categorized as full agonists, partial agonists, antagonists, and mixed agonist-antagonists. They also include ultra-short, short, and long-acting opioids. Full mu agonists are recommended as the most potent opioids: they include morphine, hydrocodone, hydromorphone, oxycodone, methadone, dihydrocodeine, oxymorphone, levorphanol, sulfentanil, alfentanil, propoxyphene, and fentanyl, which do not exhibit a ceiling effect with increasing dose. Most other opioids do have a ceiling effect. See, e.g., Module 2, Pain Management: Overview of Management Options, American Medical Assoc'n (December 2003), available online at www.ama-cmeonline.com/pain_mgmt/module02/04pharm/index.htm. Partial agonists include buprenorphine. Mixed agonist-antagonists include pentazocine, butorphanol tartrate, dezocine, and nalbuphine hydrochloride, which experience a dose-related ceiling effect. Other suitable opioids include meperidine and codeine.

Opioids may be administered orally, often as a controlled-release or slow-release formulation; they may also be administered parenterally, transdermally, or by suppository. Fentanyl is often administered transdermally, as a patch that delivers a dose into the skin and is readministered about every 72 hours. Frequently, intramuscular delivery of opioids is inappropriate, thus when injected they are often administered intravenously either as a bolus injection or as an infusion over a period of time.

For opioids having no ceiling effect, dosages are normally established for each subject based on the amount required to provide adequate pain relief, with due consideration given to observable side effects. For methods and compositions within the present invention that comprise an opioid, appropriate dosages can be similarly determined for each particular subject. The PCA methods mentioned above may be used. As is well known for opioids, each of which is associated with particular side-effect profiles, where one composition or method of the invention that comprises an opioid causes an adverse effect, it is often appropriate to change to a treatment within the invention that comprises a different opioid, since each opioid has a different side-effects profile.

Some of the adjuvant analgesics suitable for use in the present invention include caffeine, which is often used in combination with NSAIDs and acetaminophen, and members of the phenanthrenes, phenylheptylamines, phenylpiperidines, morphinans, and benzomorphans. Specific examples include corticosteroids such as dexamethasone and prednisone; antidepressants such as amitriptyline, desipramine; maprotiline, nortriptyline, fluoxetine and paroxetine; alpha-2-adrenergic agonists such as clonidine and tizanidine; anticonvulsants such as gabapentin, carbamazepine, phenyloin, valproic acid, clonazepam, lamotrigine, topiramate, tiagabine, and oxcarbazepine; NMDA receptor antagonists such as ketamine and dextromethorphan; orally delivered local anesthetics such as mexiletine, lidocaine and tocainide; bisphosphonates such as clodronate, pamidronate, and zoledronic acid; and miscellaneous compounds including baclofen, clonidine, calcitonin, prazosin, capsaicin, dextroamphetamine, modafinil, and methylphenidate. See, e.g., Module 2, Pain Management: Overview of Management Options, American Medical Assoc'n (December 2003), available online at www.ama-cmeonline.com/pain_mgmt/module02/04pharm/index.htm. Dosages and routes of administration for the use of each of these compounds to treat pain are well known in the art; dosages and routes of administration for their use in the methods and compositions of the present invention are thus readily determined by those of ordinary skill in the art.

Details of the methods for using transcutaneous electrical nerve stimulation (TENS), percutaneous electrical nerve stimulation (PENS), acupuncture, and related techniques to alleviate pain are well known. Textbook of Pain, 4^{th} ed., P. D. Wall and R. Melzack, ed., Elsevier Science, pp. 1341-46 (2003). See also P. F. White, et al., Percutaneous Electrical Nerve Stimulation (PENS): A Promising Alternative-Medicine Approach to Pain Management, American Pain Society, vol. 9(2) (1999) The details for using these non-pharmacological pain therapies in the methods of the invention are therefore readily determined.

Those of skill in the art will appreciate that certain aspects and embodiments of the disclosed invention can be combined, and such combinations are also within the scope of the invention. The following examples are offered to illustrate but not to limit the invention.

### EXAMPLE 1

### Evaluation of Interaction of an N-Type Calcium Channel Blocker and Morphine Against Tactile Hyperesthesia and Thermal Hyperalgesia in Rats with SNL: Isobolographic Analysis

The interaction of two drugs (an N-type blocker such as 1-(4-benzhydrylpiperazin-1-yl)-3,3-diphenylpropan-1-one and morphine) against tactile hyperesthesia and thermal hyperalgesia is determined in rats with spinal nerve ligation (SNL). Dose-response curves against each endpoint have been established for each of the compounds and the A₅₀ values (doses producing a 50% maximal effect) are known. A fixed ratio of drug is employed, based on the ratio of calculated A₅₀ values. Dose-response curves for the mixture are generated against tactile and thermal endpoints and isobolographic analysis is employed to determine if the drug combination is synergistic or simply additive. Male Sprague-Dawley rats are subjected to SNL or sham surgery and tested 10 days post-surgery. Each dose-response curve requires 4 data points (doses), and for each dose 8 rats are used. The animals are tested at 10, 20, 30, 45 and 60 minutes after injection in order to establish a time-course and time of peak effect and the dose-response curves are generated from data gathered at the time of peak effect. Separate curves for the N-type blocker plus morphine (fixed ratio) are generated against tactile hyperesthesia and against thermal hyperalgesia.

Nerve ligation injury is performed according to the method described by Kim and Chung (1992) “An experimental model for peripheral neuropathy produced by segmental spinal nerve ligation in the rat.” Pain 50 (3), 355-363. This technique produces signs of neuropathic dysesthesias, including tactile allodynia, thermal hyperalgesia and guarding of the affected paw. Rats are anesthetized with 2% halothane in O₂ delivered at 2 liters/min. The skin over the caudal lumbar region is incised and the muscles retracted. The L₅ and L₆ spinal nerves are exposed, carefully isolated, and tightly ligated with 4-0 silk suture distal to the dorsal root ganglion. After ensuring homeostatic stability, the wounds are sutured, and the animals allowed to recover in individual cages. Sham-operated rats are prepared in an identical fashion except that the L₅/L₆ nerve roots are not ligated. Any rats exhibiting signs of motor deficiency are euthanized.

Thermal hyperalgesia is determined by the method of Hargreaves et al. “A new and sensitive method for measuring thermal nociception in cutaneous hyperalgesia.” Pain. 32 (1988) 77-88. A radiant heat source is focused onto the plantar surface of the affected paw of nerve-injured or sham-operated rats. When the animal withdraws its paw, a motion sensor halts the stimulus and timer. A maximal cut-off of 40 sec is utilized to prevent tissue damage. Paw withdrawal latencies are thus determined to the nearest 0.1 sec. The withdrawal latency of sham-operated rats is compared to those of ligated rats to measure the degree of hyperalgesia. Antinociception is indicated by paw withdrawal latencies exceeding those of normal baseline values. Data are converted to % Antinociception by the formula: % Antinociception=100×(test value−control value)/(40 sec−control value).

The antinociceptive effects of combinations of drugs are analyzed for additive or synergistic interactions by isobolographic analysis (Tallarida, R. J. (1992) Statistical analysis of drug combinations for synergism. Pain 49, 93-97). Log dose-response curves for each component administered alone are established and the A₅₀ (95% C.L.) calculated. The A₅₀ (95% C.L.) for the log dose-response curve of a drug mixture at each fixed ratio is calculated in terms of “total dose” administered. For a given drug combination, a theoretical A₅₀ exists such that A_{50 add}=A_{50 drug1}×(p₁+Rp₂) where R is the potency ratio of drug 1 to drug 2, p₁ is the proportion of drug 1 in the mixture and p₂ is the proportion of drug 2. Variances and 95% C.L. for the theoretical additive A₅₀ are derived from the variances of each drug administered alone. A t-test is employed to compare the theoretical additive A₅₀ and 95% C.L. to that obtained for the mixture. A significantly lower experimental value compared to theoretical value denotes a synergistic interaction.

### EXAMPLE 2

A composition for the treatment of pain in a subject experiencing chronic pain is prepared by admixing an N-type calcium channel blocker with an NSAID. The N-type calcium channel blocker is 1-(4-benzhydrylpiperazin-1-yl)-3,3-diphenylpropan-1-one, and the NSAID is ibuprofen. The two compounds are mixed; the relative amounts of the active compounds are determined based on the dosage of each to be administered. Fillers and binders are added, and the mixture is formed into tablets sized to deliver 400 mg of ibuprofen and an effective dose of the N-type calcium channel blocker.

### EXAMPLE 3

A subject experiencing chronic pain is identified. The composition of Example 2 is provided to the subject, who is instructed to ingest one tablet every 6 hours as needed to obtain suitable pain relief.

### EXAMPLE 4

An N-type calcium channel blocker that is 6,6-diphenyl-1-[4-(3-phenyl-2-propen-1-yl)-piperazin-1-yl]hexan-1-one is formulated for oral administration and is formed into tablets, so that each tablet contains a unit dosage amount of the compound. A subject experiencing severe pain is identified. A fentanyl patch designed to provide an effective amount of fentanyl via transdermal delivery is affixed to the skin of the subject. One tablet comprising the N-type calcium channel blocker is administered to the subject every three hours during the first twelve hours. After that time, the subject is instructed to self-administer tablets of the N-type calcium channel blocker only as needed to alleviate pain. The fentanyl patch is replaced according to the recommended schedule provided by its manufacturer.

### EXAMPLE 5

A low dose of amitriptyline (one 50 mg pill per day) is administered to a subject experiencing diabetic neuropathy and nausea. An effective amount of an N-type calcium channel blocker, 6,6-diphenyl-1-[4-(cyclohexylmethylamino)-piperidin-1-yl]hexan-1-one, is administered as a suppository using standard controlled-release formulation.

### EXAMPLE 6

A subject experiencing severe post-operative pain is treated with an effective amount of an N-type calcium channel blocker, 6,6-bis-(4-fluorophenyl)-1-[4-(3,4,5-trimethoxyphenylmethyl)piperazin-1-yl]hexane, which is administered intravenously as a continuous IV drip. The patient is provided with a patient-controlled supply of morphine that can be self-administered as needed, and acetaminophen tablets are administered periodically at a rate of 500 mg every 6 hours.

1. A composition for alleviation of pain, which composition comprises a combination of a first compound that is an N-type calcium ion channel blocker of formula (1): 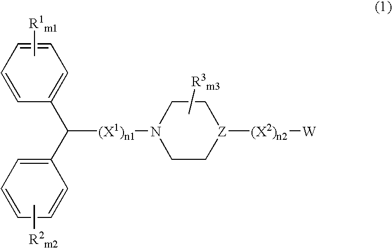
wherein each m¹ and m² is independently 0-5;
m³ is 0-2;
each of R¹, R² and R³ is independently an optionally substituted alkyl, alkenyl, alkynyl, aryl, arylalkyl, or arylalkenyl each optionally having one or more C replaced by a heteroatom selected from N, O and S wherein said optional substituents may include one or more ═O, or each of R¹, R² and R³ is independently halo, CF₃, CN, OCF₃, NO₂, NO, SO, SO₂, NR₂, OR, SR, COOR, or CONR₂ wherein R is H or optionally substituted alkyl, alkenyl, alkynyl, alkyl, arylalkyl or arylalkenyl, and wherein two of each of said R¹, R² or R³ may form a 3-7 membered saturated or unsaturated ring;
Z is N or CH;
wherein each of n¹ and n² is independently 0 or 1;
each X¹ and X² is independently an optionally substituted alkylene or alkenylene optionally including one or more heteroatoms selected from N, O and S; and
W is Ar or Cy, wherein
Ar represents one or two substituted or unsubstituted aromatic or heteroaromatic rings, and
Cy represents one or two substituted or unsubstituted aliphatic cyclic or heterocyclic moieties, or consists of one substituted or unsubstituted aliphatic cyclic or heterocyclic moiety and one substituted or unsubstituted aromatic or heteroaromatic moiety,
or a pharmaceutically acceptable salt thereof,
with a second compound that alleviates pain through a mechanism other than N-type calcium channel blockage,
wherein the combination is effective to alleviate pain.

2-3. (canceled)

4. The composition of claim 1 wherein the first compound is:
1-(4-benzhydrylpiperazin-1-yl)-3,3-diphenylpropan-1-one;
6,6-diphenyl-1-[4-(3-phenyl-2-propen-1-yl)-piperazin-1-yl]hexan-1-one;
6,6-diphenyl-1-[4-(cyclohexylmethylamino)-piperidin-1-yl]hexan-1-one;
6,6-bis-(4-fluorophenyl)-1-[4-(3,4,5-trimethoxyphenylmethyl)piperazin-1-yl]hexane; or
4-benzhydryl-piperazine-1-carboxylic acid benzhydryl-amide;
or a pharmaceutically acceptable salt thereof.

5. The composition of claim 1, wherein the first compound is 1-(4-benzhydrylpiperazin-1-yl)-3,3-diphenylpropan-1-one, or a pharmaceutically acceptable salt thereof.

6. The composition of claim 1, formulated to be administered orally, transmucosally or transdermally.

7. (canceled)

8. The composition of claim 1, wherein the second compound is an NSAID, a selective Cox-2 inhibitor, an opioid or an adjuvant analgesic.

9-11. (canceled)

12. A method to ameliorate pain in a subject, which method comprises administering to a subject in need of such amelioration a first compound that is an N-type calcium ion channel blocker of formula (1): 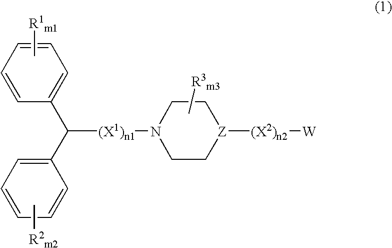
wherein each m¹ and m² is independently 0-5;
m³ is 0-2;
each of R¹, R² and R³ is independently an optionally substituted alkyl, alkenyl, alkynyl, aryl, arylalkyl, or arylalkenyl each optionally having one or more C replaced by a heteroatom selected from N, O and S wherein said optional substituents may include one or more ═O, or each of R¹, R² and R³ is independently halo, CF₃, CN, OCF₃, NO₂, NO, SO, SO₂, NR₂, OR, SR, COOR, or CONR₂ wherein R is H or optionally substituted alkyl, alkenyl, alkynyl, alkyl, arylalkyl or arylalkenyl, and wherein two of each of said R¹, R² or R³ may form a 3-7 membered saturated or unsaturated ring;
Z is N or CH;
wherein each of n¹ and n² is independently 0 or 1;
each X¹ and X² is independently an optionally substituted alkylene or alkenylene optionally including one or more heteroatoms selected from N, O and S; and
W is Ar or Cy, wherein
Ar represents one or two substituted or unsubstituted aromatic or heteroaromatic rings, and
Cy represents one or two substituted or unsubstituted aliphatic cyclic or heterocyclic moieties, or consists of one substituted or unsubstituted aliphatic cyclic or heterocyclic moiety and one substituted or unsubstituted aromatic or heteroaromatic moiety,
or a pharmaceutically acceptable salt thereof, and
administering to said subject a second compound that ameliorates pain by a mechanism other than N-type calcium channel blocking,
wherein said compounds are administered in amounts which in combination are effective to ameliorate said pain.

13-14. (canceled)

15. The method of claim 12 wherein the first compound is:
1-(4-benzhydrylpiperazin-1-yl)-3,3-diphenylpropan-1-one;
6,6-diphenyl-1-[4-(3-phenyl-2-propen-1-yl)-piperazin-1-yl]hexan-1-one;
6,6-diphenyl-1-[4-(cyclohexylmethylamino)-piperidin-1-yl]hexan-1-one;
6,6-bis-(4-fluorophenyl)-1-[4-(3,4,5-trimethoxyphenylmethyl)piperazin-1-yl]hexane; or
4-benzhydryl-piperazine-1-carboxylic acid benzhydryl-amide;
or a pharmaceutically acceptable salt thereof.

16. The method of claim 12 wherein the first compound is 1-(4-benzhydrylpiperazin-1-yl)-3,3-diphenylpropan-1-one, or a pharmaceutically acceptable salt thereof.

17-18. (canceled)

19. The method of claim 12, wherein the second compound is an NSAID, a selective Cox-2 inhibitor, an opioid or an adjuvant analgesic.

20-22. (canceled)

23. A method to alleviate pain in a subject, which method comprises administering to the subject an N-type calcium ion channel blocker of formula (1): 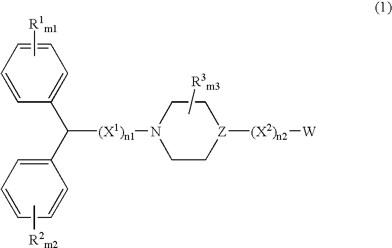
wherein each m¹ and m² is independently 0-5;
m³ is 0-2;
each of R¹, R² and R³ is independently an optionally substituted alkyl, alkenyl, alkynyl, aryl, arylalkyl, or arylalkenyl each optionally having one or more C replaced by a heteroatom selected from N, O and S wherein said optional substituents may include one or more ═O, or each of R¹, R² and R³ is independently halo, CF₃, CN, OCF₃, NO₂, NO, SO, SO₂, NR₂, OR, SR, COOR, or CONR₂ wherein R is H or optionally substituted alkyl, alkenyl, alkynyl, alkyl, arylalkyl or arylalkenyl, and wherein two of each of said R¹, R² or R³ may form a 3-7 membered saturated or unsaturated ring;
Z is N or CH;
wherein each of n¹ and n² is independently 0 or 1;
each X¹ and X² is independently an optionally substituted alkylene or alkenylene optionally including one or more heteroatoms selected from N, O and S; and
W is Ar or Cy, wherein
Ar represents one or two substituted or unsubstituted aromatic or heteroaromatic rings, and
Cy represents one or two substituted or unsubstituted aliphatic cyclic or heterocyclic moieties, or consists of one substituted or unsubstituted aliphatic cyclic or heterocyclic moiety and one substituted or unsubstituted aromatic or heteroaromatic moiety,
or a pharmaceutically acceptable salt thereof
and concurrently administering to the subject transcutaneous electrical nerve stimulation (TENS) or percutaneous electrical nerve stimulation (PENS).

24. The method of claim 23, wherein the N-type calcium channel blocker is selected from the group consisting of:
1-(4-benzhydrylpiperazin-1-yl)-3,3-diphenylpropan-1-one;
6,6-diphenyl-1-[4-(3-phenyl-2-propen-1-yl)-piperazin-1-yl]hexan-1-one;
6,6-diphenyl-1-[4-(cyclohexylmethylamino)-piperidin-1-yl]hexan-1-one;
6,6-bis-(4-fluorophenyl)-1-[4-(3,4,5-trimethoxyphenylmethyl)piperazin-1-yl]hexane; or
4-benzhydryl-piperazine-1-carboxylic acid benzhydryl-amide;
or a pharmaceutically acceptable salt thereof.

25. The method of claim 23, wherein the N-type calcium channel blocker is 1-(4-benzhydrylpiperazin-1-yl)-3,3-diphenylpropan-1-one, or a pharmaceutically acceptable salt thereof.

26-42. (canceled)
